(19) United States
(12) Patent Application Publication
(10) Pub. No.: US 2016/0120133 A1
(43) Pub. Date: May 5, 2016

(54) System for Plant Development

(71) Applicants: SMITHERS-OASIS COMPANY, Kent, OH (US); CLEMSON UNIVERSITY, Anderson, SC (US)

(72) Inventors: Jeffrey Adelberg, Clemson, SC (US); Jacqueline Naylor-Adelberg, Clemson, SC (US); Vijay Rapaka, Stow, OH (US)

(21) Appl. No.: 14/895,104

(22) PCT Filed: Jun. 3, 2014
(86) PCT No.: PCT/US2014/040631

§ 371 Date: Dec. 1, 2015

Related U.S. Application Data
(60) Provisional application No. 61/830,216, filed on Jun. 3, 2013.

(51) Int. Cl. A01G 9/02; A01G 9/10; A01G 1/00

(52) U.S. Cl. CPC A01G 9/021; A01G 1/001; A01G 9/1086

(57) ABSTRACT

Plant propagation systems are described that includes a flexible, self-standing container and a support that can be held within the container during one or more of sterilization, storage, shipping, and use of the system.

### CROSS REFERENCE TO RELATED APPLICATION

The present application claims filing benefit of U.S. Provisional Patent Application Ser. No. 61/830,216 having a filing date of Jun. 3, 2013, which is incorporated herein in its entirety.

### BACKGROUND

Tissue culture is used for plant propagation and genetic transformation. Tissue culture propagation, sometimes referred to as micropropagation, is the process of growing new plants from plant tissue that has been extracted from a parent plant. Horticulturists favor micropropagation as a growing method because it provides relatively high production efficiency, cleanliness and greater uniformity of plants. The process results in mass production of plants having certain desirable characteristics as substantially all of the plants produced are genetically identical to and have the desirable traits of the parent plant.

With genetic transformation, foreign DNA is introduced into a plant's genome by plasmids during co-culture, for instance via an Agrobaterium vector or particle bombardment. After selecting for successfully transformed tissue, cells regenerate shoots and root. Alternatively, transformed tissue may form bipolar embryos and then develop into plants. The transformed plantlets must be handled much like micropropagated plantlets to be brought to sexual maturity and used in breeding programs.

Micropropagation is generally described according to separate phases each of which includes growth stages. Phase I generally includes those stages in which the plant tissue is primarily heterotrophic. Stage I comprises initiation, in which explanted donor tissue is initiated in a growing media. Stage II comprises a multiplication phase in which nutrients and hormones are provided to enable rapid cell division and substantial growth of multiple plants from the explant.

In genetic transformation, Stage I explants such as immature seed embryos, or young leaf tissue are disinfested and introduced to a tissue culture medium, as in micropropagtion. In stage II of transformation, the tissues are induced to callus during co-culture with the vector, e.g., Agrobacterium, containing the foreign DNA. Following, the transformed cells are selected by transfer to a medium that would be toxic to any plant cells that did not integrate the foreign DNA. Regeneration and propagation of stage II tissues results in shoots that contain the successfully introduced foreign DNA.

In these early stages for both systems, it is very important to keep pathogens and biological pests from infesting the culture. Accordingly, the culture is generally held in an environment that shields the maturing plant from pathogens while also facilitating rapid and vigorous growth. In the first two stages of growth there are high metabolic requirements for energy consumption, but the plant tissue is not generally capable of carrying out adequate photosynthesis to meet this high demand for energy. Thus, these stages are accomplished heterotrophically. During Phase I, the plant tissue is typically exposed to adequate light intensity to signal chlorophyll development and organic carbon is obtained from sugar such as sucrose that is provided in a growth media.

In Stage III during the second phase of development, leaves and shoots expand and the plantlet becomes more photoautotrophic. During the latter phase the plant tissue derives energy when exposed to light, gases, water and essential nutrients through the process of photosynthesis. In Stage IV, the plant can be matured, often in a greenhouse, and the plant may begin to take on larger amounts of light and heat, developing roots that will be needed for transfer, for instance to a field, greenhouse or the natural environment.

The delicate state of the plant tissue, particularly in the early stages, has led to difficulties in successfully carrying out micropropagation on a large scale. For instance, the nutrients required during the early stages of growth are easily targeted by microorganisms that can destroy the young plantlets. Moreover, transplanting the plantlets from one growth media to another and/or from one growth environment to another between stages will often damage the developing plant tissue, leading to slower growth and development or even plant destruction.

One particularly difficult transition is when rooted plantlets are removed from gelled medium that is often utilized in early stage development as the soft roots are easily broken. In addition, the roots must be rinsed free of sugar-containing medium so they do not rot in soil, and the process of planting rooted plantlets in soil is tedious. Often the roots are cut off of tissue-cultured plantlets and the plantlets (micro-cuttings) are forced to root in soil under mist in a shaded greenhouse. Although this is damaging to the plantlet, economics forces require the use of micro-cuttings to be a preferred practice.

Attempts have been made to mitigate such problems. For instance, systems have been designed for maintaining the developing plant tissue in an isolated environment during early stage development. Unfortunately, many of these systems utilize hard plastic or glass containers for developing the plantlets, and there are disadvantages with the use of such containers as they are relatively heavy, and therefore typically are very costly to ship from the laboratory to a greenhouse. Further, these types of containers must be re-used many times to make their use economically viable. Re-use requires that the containers be shipped back to the laboratory as well as washed and sterilized before re-use, adding to their expense. Storage also presents a problem, as the containers are not compressible (and sometimes not even stackable) which requires a large volume of space for storage. Moreover, none of these rigid systems integrate the tissue-cultured plantlet with the transitions necessary for stage IV autotrophic growth.

Other systems have been designed that utilize flexible polymeric containers for early stage micropropagation. Problems still exist with these systems, however. For instance, gelled media utilized in the containers may lose integrity or liquid media in the base of the container can pool and waterlog developing root systems. Also, moving the plantlets from one container to another so as to provide the desired environment as the plantlets mature can damage or destroy the developing tissues. Moreover, in order to prevent infection of the developing plantlets, all of the components of a propagation system should be sterilized, and materials that could be otherwise useful in forming a flexible container system often do not hold up well to sterilization procedures. In addition, the flexibility of such containers leads to difficulties in storing and transporting the plants, for instance necessitating specially designed storage racks to secure the containers and the plants in the proper orientation.

What is needed in the plant propagation industry is a method and system for producing plants in a manner that addresses such problems. For instance, a sterilizable system that provides for light transmission and a steady nutrient supply throughout early stage development without the need for transplanting of the developing plant tissue to multiple successive culturing containers is desired. Furthermore, a system and method of safely transporting large numbers of plants easily, reliably, and at a minimum cost is needed. Additionally, what are needed are systems that can better integrate the laboratory with the greenhouse nursery.

### SUMMARY

According to one embodiment, disclosed is a system for plant development. The system can include a container and a support within the container. The container can include a base having a flat outer surface such that the container is self-standing. The container can also have a flexible wall that is semi-permeable. The entire container, e.g. the container wall and the base can be impermeable to biological microorganisms. The support can be formed of a rigid or semi-rigid material of fixed dimension that can support developing plant tissue.

A method of plant development is also described. The method can include sterilizing the plant development system. Beneficially, the sterilization can take place with the support held in the container, which can protect the support from damage during sterilization and prevent deformation of the container while maintaining the overall stability of the system. A method can also include placing plant material in or on the support, the plant material including totipotent plant cells, and sealing the container with the plant material held within the container and in or on the support. Following, the system can be maintained under conditions conducive to the growth and development of the plant material held within the system.

### BRIEF DESCRIPTION OF THE DRAWINGS

A full and enabling disclosure, including the best mode thereof, to one of ordinary skill in the art, is set forth more particularly in the remainder of the specification, including reference to the accompanying Figures, in which:

FIG. 1 illustrates a flexible, gusseted container as may be utilized in a system as disclosed herein.

FIG. 2 illustrates the top section of a container including a vent.

FIG. 3 illustrates the top section of another container including a vent.

FIG. 4 illustrates a support as may be incorporated in a system as disclosed herein.

FIG. 5 illustrates a composite system as disclosed herein.

FIG. 6 illustrates a system following addition of plant material to the system and sealing of the system.

FIG. 7 illustrates the top section of a system including a vent and a pressurization port.

FIG. 8 illustrates an Oasis® Horticubes® XL foam block support utilized in the example section.

FIG. 9 illustrates several plant growth systems as described herein and utilized in the example section.

FIG. 10 illustrates the contents of a system as disclosed herein following six weeks of laboratory plant growth.

FIG. 11 compares the rosette diameters of plants grown in various systems as described herein.

FIG. 12 compares the hyperhydricity of plants grown in various systems as described herein.

FIG. 13 illustrates the removal of liquid tissue culture medium from the foam support of a system following a growth period in a system as disclosed herein.

FIG. 14 illustrates a single cell of a support following cultivation of a plantlet in the cell at the time of transfer to greenhouse medium.

FIG. 15 illustrates the greenhouse growth ratio for plants grown in various systems as described herein.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

As used herein, the term “heterotrophic” generally refers to plant tissue, including cultured somatic embryos, that is incapable or at most weakly capable of photosynthesis. Heterotrophic plant material requires an extraneous source of carbon such as sucrose that is provided in a growth medium to provide energy and maintain normal growth and development at a desired rate.

As used herein, the term “autotrophic” with regard to plant material generally refers to plant tissue that is capable of photosynthesis. As a result, external energy-supplying compounds are not required in order for autotrophic plant material to sustain a normal growth rate.

As utilized herein, the term “callus” generally refers to a mass of unorganized and undifferentiated totipotent plant cells which, at least at a macroscopic level, are either unconnected or loosely connected, generally arising from culturing of an explant.

As utilized herein, the term “explant” generally refers to a piece of plant tissue excised from a donor plant for culturing in vitro as the source of cultured plant tissues.

As utilized herein, the term “plantlet” generally refers to a small plant with a shoot and root pole, but more immature than a seedling. A plantlet is usually heterotrophic, but may also be autotrophic.

As utilized herein, the term “somatic embryo” generally refers to a plant embryonic structure arising from an explanted somatic tissue, zygotic embryo or other totipotent plant tissue.

As utilized herein, the term “zygotic embryo” generally refers to a plant embryo that has developed directly from the zygote produced from the sexual fusion of gametes. For example, the embryo found in a seed is a zygotic embryo.

As utilized herein, the term “bud” generally refers to an organized mass of various plant tissues from which a shoot or flower will develop.

As utilized herein, the term “meristem” generally refers to a group of undifferentiated plant cells that divide to form more meristematic cells as well as somewhat differentiated cells capable of elongation and further development into plant organs and structures.

As utilized herein, the term “seedling” generally refers to a plant developed from a germinating seed to autotrophic growth.

As utilized herein, the term “embling” generally refers to a plantlet grown from a somatic embryo that is sufficiently developed for transplantation into soil.

As utilized herein, the term “rigid” refers to a material is not flexible.

As utilized herein, the term “semi-rigid” refers to a material that exhibits an amount of flexibility in that a surface of the material can deform from the original shape (e.g., planar) by an amount without cracking or breaking. For instance, upon deformation of a surface of a semi-rigid material from the original shape to an angle of greater than about 10°, 15°, or 20° from the original orientation of the surface, the semi-rigid material can crack or break. However, at a lower deformation angle, the material will not crack or break.

The present disclosure is generally directed to a plant propagation system that can protect developing plant tissue from microorganisms while supporting the tissue so as to allow for growth and development of a shoot and root without the need to transplant the plantlet in the early stages of development. The system is an inexpensive, single-use, multi-component system that can be assembled prior to sterilization and then sterilized in a single step that prevents damage and deformation of the components of the system. Moreover, the system can be efficiently shipped and stored without damage to system components both prior to and following location of developing plant tissue within the system. For example, the system can be utilized to transport large quantities of developing plants while preventing damage and maintaining sterility of the plantlets held in the system. As such, the system can be used to facilitate transportation of plant material to any desired location. For instance, some material is prohibited from international commerce due to accidental inclusions of biological contaminants with plant tissue or media. As plants held in the system can be maintained free from biotic contaminants and held on sterile substrata, quarantine regulations can be satisfied with plants transported internationally by use of the disclosed systems.

The system includes a flexible, self-standing container and a support that can be held within the container during one or more of sterilization, storage, shipping, and use (i.e., plant development) of the system. These two components can together provide many benefits to the system. For instance, the self-standing container can protect the support held inside from damage during sterilization, storage, shipping, and during plant development. Meanwhile, the support can provide an excellent growth and development medium for the plant tissue held in the system, can prevent deformation of the container during sterilization, shipping, etc., and can improve stability of the system during shipping, storage, and growth of plants. Moreover, the support can maintain the developing plant tissue prior to, during, and following development of the root structure, and the developing plant can be transplanted following the desired level of maturation in conjunction with (without removal from) the support, so as to prevent damage to the young plant at transplant. The development period for the plant within the system can include a period of growth under natural light when the plantlet or embling is protected from both microorganisms and desiccation. For example, a somatic embryo can be located in a system, developed into a photosynthetic seedling, and transplanted without ever being removed from the support.

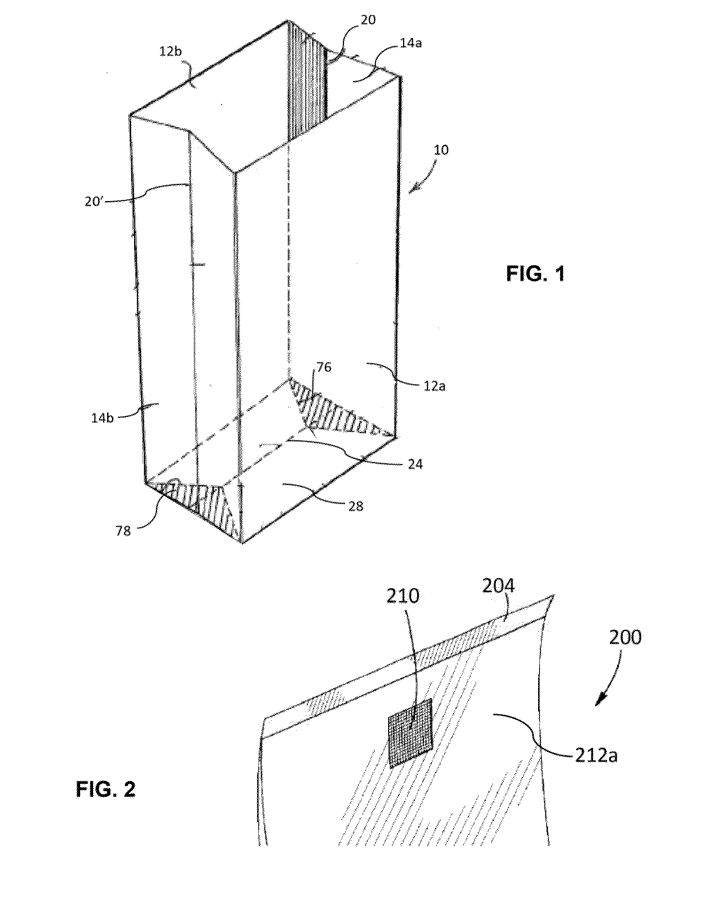

FIG. 1 shows one embodiment of a container 10 as may be utilized in a system. The container is shaped to include a front wall 12a and a back wall 12b, and side walls 14a, 14b respectively. Though illustrated with a rectangular cross section, this is not a requirement of containers of the disclosed systems, and other cross sections such as square, oval, round, etc. are encompassed herein. The container 10 also includes a base 28 that has a generally flat outer surface in order that the container 10 is capable of self-standing when placed on the base 28. For example, a gusset 76 and gusset 78 can be provided on each side of the base 28 to hold the base 28 in a flat orientation at the outer surface of the base 28. The surface area of the base is not particularly limited, and can be of a size such that the system can be stable with in use. For instance, the container base 28 can have a surface area of from about 2 square centimeters (cm²) to about 500 cm², from about 10 cm² to about 450 cm², from about 25 cm² to about 300 cm², or from about 50 cm² to about 200 cm². Likewise, the internal volume of the container 10 can vary. For example, the container 10 can have a total internal volume of from about 10 cm³ to about 1 m³, from about 50 cm³ to about 5000 cm³, from about 100 cm³ to about 3000 cm³, or from about 500 cm³ to about 2000 cm³.

Container 10 can include fold lines 20, 20′ on each side 14a, 14b, as well as a fold line 24 on the base. Fold lines 20, 20′, 24 can allow for convenient storage of the systems when not in use as the container can be folded and stacked with other containers prior to combination with the support of the system.

In one embodiment, container 10 can be formed from a single polymeric member, for instance an extruded tubular polymeric member that can be sealed at one end, for instance along fold line 24 via, e.g., a heat seal or by use of an adhesive. Following, the sealed end of the extrudate can be folded to form gussets 76, 78 according to known methodology. The gussets 76, 78 can then be adhered to the base 28, for instance by a heat seal or alternatively by use of a suitable adhesive. When an adhesive is used in formation of the container 10, the adhesive should be one that can withstand the sterilization procedures, described at more length below, and one that will not damage plant material held in the container via, e.g., leaching of the adhesive into growth media held in the container during plant development.

At least one of the container walls can be formed of a polymeric material that is semi-permeable while being impermeable to biological contaminants. More specifically, and with reference to FIG. 1, it should be understood that while in one embodiment the container 10 can be formed of a single polymeric member, this is not a requirement, and in other embodiments multiple different materials can be used in forming the container 10. In any case, however, the walls 12a, 12b, side walls 14a, 14b, and base 28 of container 10 can be flexible, and all of the material(s) forming container 10 can be liquid impermeable and impermeable to biological contaminants. As such, plant material enclosed within the sealed container 10 can receive light and meet respiratory needs of the developing plant tissues while remaining shielded against pathogenic microorganisms.

The wall(s) of the container can be liquid impermeable while allowing for respiration of the developing plant tissue held within the container. For example, in one embodiment, at least one wall of the container 10 can have a permeability to carbon dioxide (CO₂) that is equal to or greater than about 100 cubic centimeters (cc) per 100 square inches (in²) per 24 hours (h) at 1 atmosphere (atm) pressure or greater. For example, the CO₂ permeability can be from about 200 to about 1200 cc/100 in²/24 hours at 1 atm. The container wall(s) can have a permeability to oxygen (O₂) of equal to or greater than about 100 cc/100 in²/24 hours at 1 atm, for instance from about 100 to about 450 cc/100 in²/24 hours at 1 atm. The moisture vapor transmission rate (MVTR) of the container (i.e., at least one wall of the container) can generally be equal to or less than about 1 gram (g)/100 in²/24 h at 1 atm. For example, the MVTR can be from about 0.2 g/100 in²/24 hours at 1 atm to about 0.7 g/100 in²/24 hours at 1 atm.

In addition, in order to shield the interior of the container from pathogenic organisms, the walls 12a, 12b, 14a, 14b, and base 28 can have a low porosity, so as to block pathogenic microorganisms from entry. For example, the walls 12a, 12b, 14a, 14b, and base 28 can generally have a pore size of about 0.2 micrometers (μm) or less, about 0.1 μm or less, or about 0.05 μm or less, for preventing the passage of pathogenic microorganisms.

The thickness of the walls 12a, 12b, 14a, 14b and base 28 can vary, but can generally be from about 1.0 mil to about 4.0 mils, for instance about 2.0 mils. If the membrane material is much thinner than about 1.0 mil, handling of the container may be more difficult, as the opposing walls 12a, 12b, 14a, 14b may adhere to each other. Of course, this may vary depending upon the specific material used to form the container 10 and in one embodiment the wall(s) may be thinner than about 1.0 mil. Beneficially, the relatively thin, translucent walls of a container 10 can provide excellent clarity and permit viewing of a tissue sample enclosed in the container, for instance by the laboratory staff. For example, the walls of a container may be thin and translucent as compared to wall materials of many prior art containers such as glass containers. The thicker structure of many previously known containers often hinders visibility, which can affect decision making regarding plant care as well as complicating the storage and transport of these heavier, bulkier containers.

The walls 12a, 12b, 14a, 14b of the container 10 can be translucent so as to allow the passage and diffusion therethrough of light rays having at least the wavelengths of from about 400 nanometers (nm) to about 750 nm. Wavelengths in this range are required by individual photosynthetic agents, such as the chlorophylls, in green tissue plants to provide the reactions necessary for life and growth.

The materials used to form the container 10 can also be capable of withstanding a sterilization procedure. For instance, the container 10 can be resistant to high temperature and high pressure sterilizing treatments such as autoclave treatment conditions including subjection to high pressure saturated steam at temperatures of from about 120° C. to about 140° C. and at a pressure of from about 15 pounds per square inch (psi) to about 30 (psi).

In general, any polymeric material that meets the desired characteristics including permeability and ability to withstand the heat and pressure of a sterilization procedure can be utilized in forming the container 10. By way of example, a high density polyethylene or a polypropylene can be utilized to form the container 10. For instance, a polyethylene having a density of from about 0.93 g/cm³ to about 0.97 g/cm³ can be utilized in forming the container. In one embodiment, at least one wall of the container can be formed of biaxially oriented polypropylene.

A biaxially oriented polypropylene is stretched in both the machine and cross directions so as to increase the strength and clarity of the material. According to one embodiment, a sequential biaxial orienting method can form a film that can be utilized in forming a container 10. By way of example, pellets or chips of a polypropylene resin can be supplied to an extruder and then heated and melted at a temperature of about 170° C. to about 320° C. The melt is extruded from a die and then cooled and solidified, for instance on a metal drum held at a temperature of from about 60° C. to about 140° C. to obtain a cast raw sheet with β-form crystals. Next, the cast raw sheet is made to pass between rolls rotating at different rates while maintaining the temperature of the cast raw sheet from about 100° C. to about 160° C. to stretch the cast raw sheet in the flow direction and gain, e.g., a three- to seven-fold length increase. After that, the resultant sheet is cooled. Following, the cooled sheet is directed to a tenter, and stretched in the width direction to gain, e.g., a three- to eleven-fold width increase while keeping the temperature of the sheet at about 150° C. or more. Finally, the resultant biaxially oriented sheet is relaxed and subjected to thermal fixing, followed by winding and further processing, for instance to form the container 10.

The container can optionally include a vent in a wall. For example, container 200 illustrated in FIG. 2 includes a gas diffusion membrane vent 210 that can facilitate diffusion of gases from the outer surface of the container to the inner space of the container. Of course, the gas diffusion membrane vent 210 can have any suitable shape including round, oval, rectangular, etc. and is not limited to a square vent 210 as illustrated in FIG. 2. During use, oxygen, nitrogen, carbon dioxide, etc. can pass through the vent 210. The membrane vent 210 can be formed of materials similar to those of container 200, with the difference that the membrane vent allows a larger diffusion of gases as compared to the container. For example, the membrane vent 210 can be formed of a high density polyethylene or a polypropylene that is liquid impermeable and impermeable to pathogenic agents with an increased permeability to oxygen and carbon dioxide as compared to the container 200.

In the embodiment of FIG. 2, a gas diffusion membrane vent 210 is provided upon the first wall 212a of the container. The container 200 can also be formed such that sealed edge 204 may be folded over the membrane vent 210 to better control the gas diffusion characteristics of the system. For example, after the desired plant tissue has been located within the system, the system may be filled with the correct gasses for healthy plant growth and sealed.

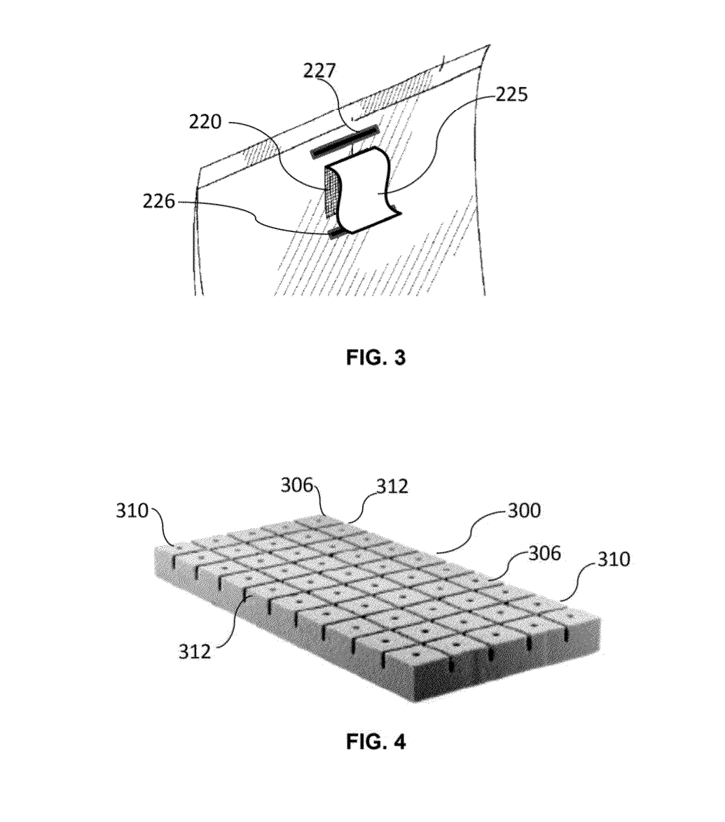

Alternatively, a vent may be open throughout use of the device with no closure or may have a separate closure. For instance, in the embodiment illustrated in FIG. 3, the container can include a vent 220 that incorporates a closure 225. As can be seen, closure 225 is in the form of a flexible flap that can overlay the vent 220 as desired to effectively close the vent 220 and prevent diffusion through the vent 220. In this embodiment, closure 225 can be attached to the container when vent is to be closed as with adhesive strip 226. The container can also include a second adhesive strip 227 that can be used to hold the closure 225 such that vent 220 is uncovered and open.

Referring again to FIG. 2, during certain times of development of plant tissue held within the system, the membrane vent 210 can be uncovered and the developing plant tissue can receive additional gasses in the controlled environment. For example, utilization of a membrane vent can reduce the development of hyperhydricity in the growing plants. During transport of the system the vent can be closed, e.g., covered by sealed edge 204 or some other closure device to avoid gas exchange between the container interior and the surrounding environment during transport.

To affix the membrane vent 210 to the container, a slice or hole can be formed in the container and the membrane vent can be affixed to the container over the hole via, e.g., a heat seal, a suitable adhesive, etc.

FIG. 4 illustrates a support 300 as may be incorporated in a system in conjunction with a container. Support 300 can be formed of a self-supporting rigid or semi-rigid cohesive material having fixed dimensions that can support developing plant tissue, can withstand sterilization procedures in conjunction with the container and prevent deformation of the container during the sterilization, and can provide form and stability to the flexible container.

In one embodiment, support 300 can be a polymeric foam that can be utilized as a substrate for plant growth. This is not a requirement, however, and support 300 may be formed of other materials that can provide a rigid or semi-rigid support of fixed dimension. For instance, support may be formed of a natural material that can include a natural binder or that is held together with a natural or synthetic binder. Examples of natural materials include mineral wool materials, such as rockwool or glass wool or Jiffy® Preforma®, which is formed of a natural material adhered with a synthetic polymer.

Support 300 can be a composite that can include an open-celled foam composition in conjunction with other materials. For instance, support 300 can be at least in part formed of a foam made from a phenolic, polyurethane, latex, urea-formaldehyde, or polyisocyanurate-based homopolymer or copolymer, with phenolic-based foam being utilized in one embodiment. The foam or natural material may be used in conjunction with one or more filler materials, such as peat. Materials for use as a support 300 are available on the retail market, for instance from Smithers-Oasis Company of Kent, Ohio. By way of example, Oasis® Horticubes® XL seed propagation medium can be utilized to form support 300. A foam support can be preferred in one embodiment as use of a foam support can make transplanting the grown plant easier, as a section of foam (e.g., a foam cube) can be easily broken off from the remainder of the support and the resulting element can then be directly transplanted into the soil. Other support materials may require that the support material be separated from the roots before transplanting, which can cause root damage and can make transplanting more difficult. A foam support such as the Horticubes® XL foam product breaks apart, allowing the roots to be washed of the growth medium without damage to the roots.

In one embodiment, the support can be pre-formed and then placed within the container. Alternatively, the support can be formed in situ. For instance, the support can be a porous polymeric material that can be cast as a liquid (e.g., a solution or dispersion) and then can cure within the container to form the support having fixed dimensions within the container.

The support 300 can have dimension stability and be self-supporting and rigid or semi-rigid. The material used as support 300 can be absorbent, but can also have water holding capacity, in that it releases water as well as absorbs it, making water available to the plant. It can be capable of withstanding conditions encountered during sterilization, such as by autoclaving. One advantage of a foam material is that it can cause minimal or no damage to the plant roots when the plant is removed from the material. Moreover, a foam material has a cellular structure providing air porosity that aids in the exchange of oxygen to the developing root structure. The support 300 may also be capable of being segmented into individual cells 310, as hereinafter described.

A support 300 can be designed with a plurality of individual cells 310, each of which can support a developing plant. The cells can be separable from one another, which can aid in transplant of the plantlets following initial development utilizing the disclosed system. For instance, in the illustrated embodiment of FIG. 4, the cells 310 can be separated from one another by a score 312, along which the cells 310 can be broken apart at the time of transplant. In addition, each cell 310 can include an opening 306 formed therein, and the plant tissue can be initially located within the opening 306 of the support 300 for growth and development.

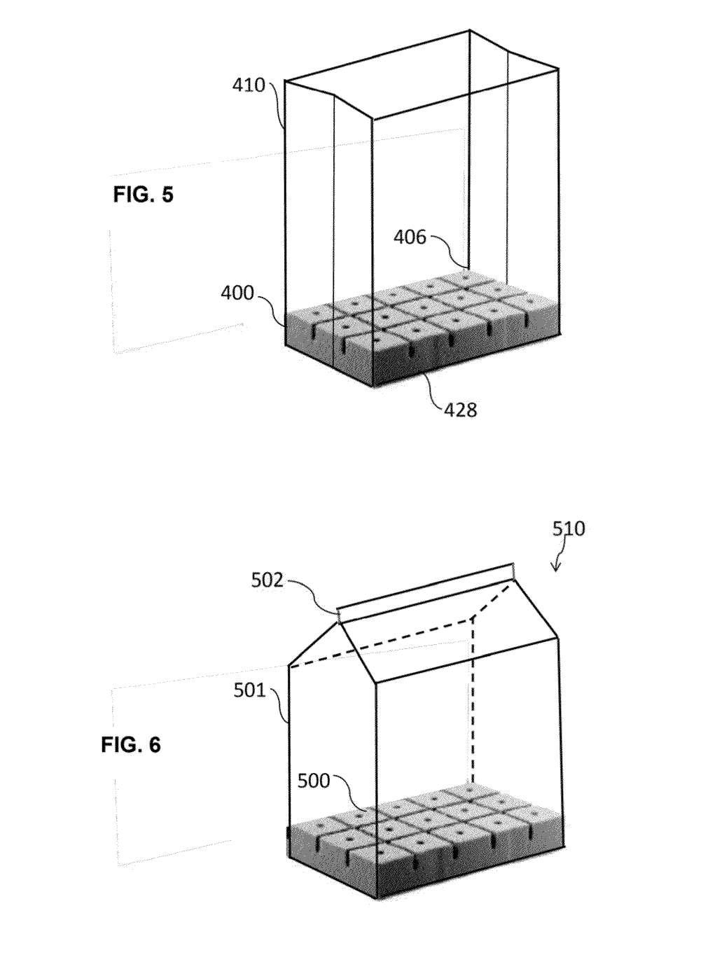

As shown in FIG. 5, upon assembly, a system can include a support 400 held within a container 410. The support can be sized so as to generally fit the dimensions of the base 428, which can improve stability of the system as well as prevent distortion of the container 410 during sterilization of the system. For instance, the support can have dimensions that are from about 95% to about 99.9% of the dimensions of the base 428 of the container and the support can thus be nested within the container base 428 and provide additional stability to the free-standing container 410 and the system.

Following assembly, the entire system can be sterilized, for instance by use of an autoclave, according to standard practice. By way of example, an autoclave sterilization process may be utilized during which the autoclave may reach a temperature of about 250° F. (about 120° C.) at a pressure of about 15 psi.

An aqueous growth medium can be included in the system either prior to or following sterilization. Addition of the aqueous growth medium prior to sterilization may be preferable in some embodiments, as the sterilized system can be stored and shipped ready for use, and the end user need only insert the desired plant tissue (e.g., a callus, a microcutting, meristemic cells, etc.) into an opening 406 of the support 400 and close the top of the container. The top of the container 410 can be held in a closed arrangement either with a tight seal or a loose closure. For instance, the container top can be closed with a heat seal for a tight seal or more loosely, as with a clip, for a loose seal.

A growth medium can generally be any suitable medium as is known in the art, with preferred media depending upon the plants to be developed within the system as well as the growing conditions to be utilized during the time the plants are held in the system. By way of example, a growth media can include, without limitation, one or more of NH₄NO₃, KNO₃, H₃BO₃, KH₂PO₄, KI, Na₂MoO4.H₂O, CoCl₂.6H₂O, CaCl₂.2H₂O, MgSO₄.7H₂O, MnSO₄.H₂O, ZnSO₄.7H₂O, CuSO₄.5H₂O, pyridoxine nicotinic acid, glycine, sucrose, and so forth. Plant growth media as are known in the art can be utilized such as, for example, micropropagation medium as described by Murashige and Skoog (MS 1962), supplemented with 3% sucrose, organic components as described by Linsmaeier and Skoog (1965), with 1 μM of a plant growth regulator such as meta-topolin. A plant growth media can be at a pH of between about 5 and about 7, for instance at pH 5.7. Of course, desirable pH of a media can depend upon the specific plant tissue to be developed by use of the system.

A medium containing mineral nutrients can be utilized in promoting the growth of the heterotrophic plants, but is not necessary in all cases. In addition, the nutrients and carbon and energy source may be mixed in dry powder or particulate form and thereafter water can be added to form the aqueous medium. The term aqueous solution or medium thus encompasses a solution formed by adding water to a support that contains particulate nutrients and other materials as well as a solution formed by mixing such materials with water and applying the formed mixture to the support.

The aqueous medium may also include one or more plant growth hormones to stimulate growth and development of plant structures, such as shoots or roots, from the plant tissue supplied to the system. While somatic embryos may have sufficiently developed rudimentary shoot and root so as to not require growth hormones in the medium, other types of heterotrophic plant material may not, such as micropropagated adventitious meristematic tissue, buds, or microcuttings. Hence, depending upon the particular type and state of development of the heterotrophic plant material embedded in the support, the addition of plant hormones such as auxins and cytokinins may be advantageous.

The amount of aqueous media included in a system can vary depending upon the length of time the system is to be utilized, the type of plant to be developed by the system, the particular growing conditions to be utilized, etc. For example, aqueous media can be included in a system in an amount of from about 0.3 milliliters per cubic centimeter of support (mL/cm³) to about 1 mL/cm³, for instance from about 0.4 mL/cm³ to about 0.9 mL/cm³. Of course, higher or lower amounts are likewise encompassed herein, depending upon the nature of the development process.

To utilize the system, a plant material such as vegetative buds, bulbets, miocrotubers, transformed tissue, or somatic embryos can be placed within the container, for instance when the plant tissue is capable of forming shoots, but ill-suited for autotrophic growth. The plant material to be developed in the system can be a somatic embryo or developed from a somatic embryon, but the system is not limited to development of only somatic embryos, and the heterotrophic plant material may be any viable unit of living plant material containing totipotent cells capable of growing under controlled conditions into a complete autotrophic plant possessing normal roots and shoots. One source of such heterotrophic plant material is a liquid culture of plant somatic embryos that can be derived from explanted zygotic embryos of a source plant. This process, such as described by Durzan and Gupta, Plant Science 52:229-235 (1987), involves several culture steps involving different gel and liquid media containing mineral nutrients, organic compounds to supply carbon and energy, specific plant hormones, and water. Other sources of suitable heterotrophic plant material are cultured meristematic tissue, explanted zygotic embryos, cultured bud tissues, totipotent callus tissues, and the like, produced by any of a number of currently practiced plant propagation techniques including micropropagation techniques, somatic embryogenesis, plant regeneration, genetic transformation, and so forth.

After the plant tissue has been placed in the system, the container can be sealed, for instance with a heat seal as illustrated in FIG. 6 to form a closed top 502. Once sealed, the system 510 can be conveniently held either alone or in conjunction with other systems for development of the plant tissue held inside as well as for shipping of the plant tissue. For instance, a plurality of the systems 510 can be stacked and packed together for shipping by merely folding over the top 502. Upon folding the top 502, system 510 can form a stackable cube-type shape with the developing plant material protected inside. The combination of the sealed container 501, which will prevent the gases inside from escaping, and the support 500 held within the container 501 can provide a stable system that can be packed and/or stacked with other such systems without damage to the plant material held inside.

The pressure inside the sealed container 501 can increase as compared to the external pressure upon folding over the top 502 and sealing the system. The amount of folding (i.e., the decrease in internal volume of the container) can be utilized to control the pressure increase in the system.

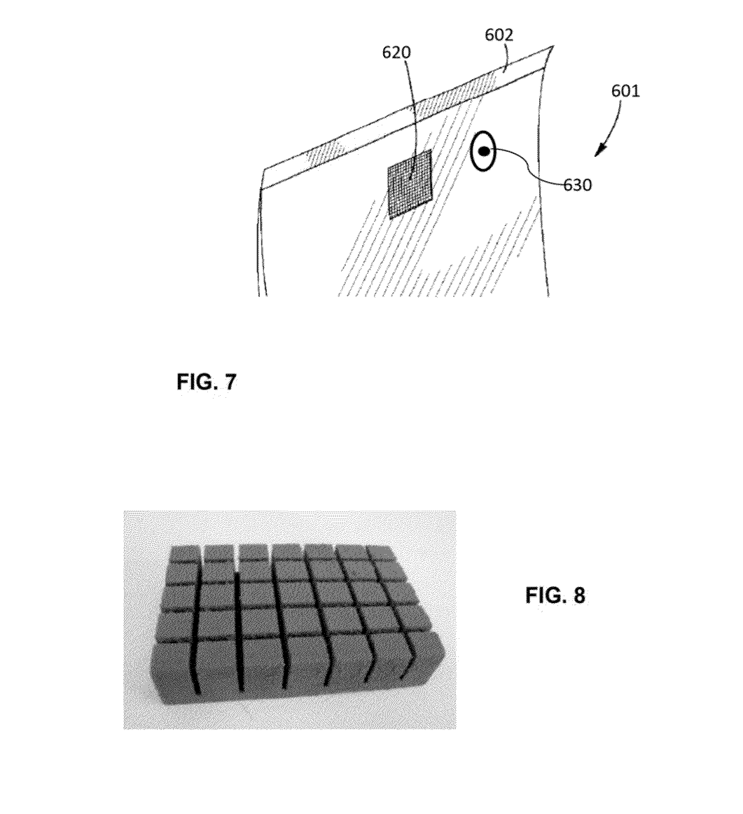

In one embodiment, illustrated in FIG. 7, the container 601 can include a vent 620 and a port 630 near the sealed top 602 of the container 601. The port 630 can be utilized to inject or remove gases into or out of the container 601. This may be utilized to control pressure within the container 601 as well as to control the gaseous contents of the container 601 during growth and development of the plant material held within the system. For instance, following a period of growth and development, an amount of the gases inside the container can be removed so as to remove excess oxygen from the container, and gas higher in carbon dioxide can be injected through the port 630. A port can be, for instance, a one-way injection port that can be utilized with a syringe, or the like.

The initial growth and development stages of the plant tissue can be carried out in a laboratory setting, for instance under a predetermined growth schedule and with controlled lighting sources. The systems can optionally be utilized for growth and development of the plant material within a greenhouse. For example, initial growth and development can be carried out in a laboratory setting, and the system can be moved to a greenhouse when the developing plant material is strong enough and further development can then be carried out in the greenhouse. Beneficially, the container of the system is water impermeable and as such the exterior of the container may be intermittently misted without diluting the growth media held in the container. Upon misting, the evaporation of water from the container's exterior surface can help to control the interior temperature of the system (for instance when the containers are utilized in a greenhouse).

Once developed into a small plant capable of surviving in soil, the plant can be transplanted. For instance, the container can be opened, and an individual cell can be broken off from the other cells of the system. The cell, which carries a young plant, may be voided of organic compounds by rinsing, and can then be transplanted in its entirety, with no need for separation of the young plant from the support, which can increase likelihood of the plants long-term survival.

The present disclosure may be further understood with reference to the examples, below.

### EXAMPLE 1

### Materials

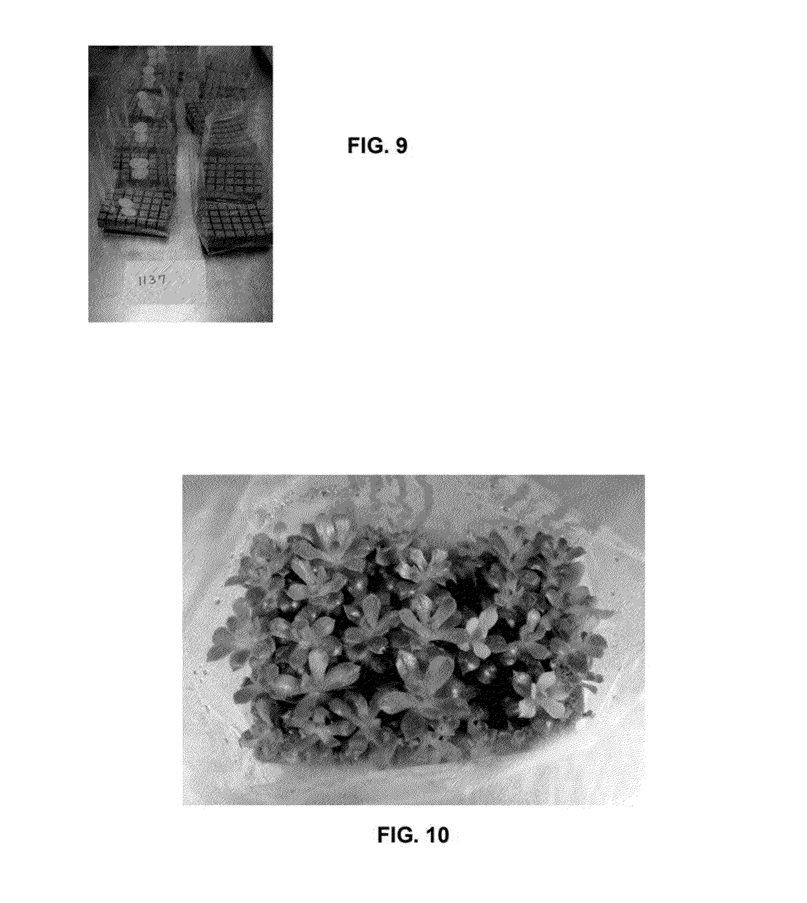

Aseptic cultures of Echeveria ‘Black Prince’were maintained in vitro on the standard micropropagation medium Murashige and Skoog (MS 1962), solidified with agar, and supplemented with 3% sucrose, Linsmaeier and Skoog (1965) organic components, with 1 μM of the plant growth regulator meta-topolin, at pH 5.7. Oasis® Horticubes® XL foam blocks of 11.75 cm×7.75 cm×2.5 cm were top grooved 2 cm deep to form 35 cells of 0.5 cm×0.7 cm. Flat-bottom gusseted containers made of clear, biaxially oriented polypropylene (BOPP; 2 mil thickness) film were used. Each 12 cm×8 cm×18 cm container was fitted with an Oasis® Horticubes® XL foam block similar to that illustrated in FIG. 8. The foam block was rinsed thrice with distilled water and air dried. Some of the systems included an air vent on the container (FIG. 9).

### Laboratory Method

Each system included one block of foam inserted into one container and infused with liquid nutrient medium (as above, without the agar solidifying agent). Each system was autoclave sterilized and then cooled with a paper clip lightly closing the container by rolling the top of the container over. The support used in the example was Oasis® Horticubes® XL, a phenolic foam growing medium with three volumes of liquid medium (100, 150, and 200 ml); and the inclusion or omission of four 1 cm ventilation patches on the containers; with two vessel system replicates for each treatment factor combination.

Microcuttings of Echeveria ‘Black Prince’were aseptically inserted into scored cells of the foam blocks. The containers were sealed with an impulse sealer three times across the top (0.5 cm between each seal). Then the sealed portion was rolled downward effectively decreasing the volume of the vessel and forcing its inflation. A paper clip held the fold in place, maintaining the 3-dimensional confirmation. Plantlets were grown in a culture room with 40 μmol cool white fluorescent light, 16 hr days, at 24° C. for 6 weeks.

### Greenhouse Method

In the greenhouse, the containers were cut open, the liquid medium was removed by soaking in water, each plant was evaluated (diameter, number of divisions, presence of hyperhydric tissue, etc.), and the foams cells were separated and planted. Greenhouse medium was a Fafard 3b soilless mix (peat, pine bark, perlite; Fafard Inc., Anderson S.C.) supplemented with 30% additional perlite. A rooted plantlet in a saturated Oasis® Horticubes® XL plug was planted in greenhouse medium in 10″×20″ greenhouse flats with 1206 cells (72 cells per flat). Plants were grown for 5 weeks in December and January under full sun, hand irrigated with 60 ppm-N 20-10-20 Peat Lite Special water soluble fertilizer (Peter's Co., Allentown Pa.). Following greenhouse culture, plants were evaluated (survival, diameter, number of divisions).

### Statistical Analysis

The experiment was a completely randomized design. ANOVA was conducted on all quantitative data for the full factorial of media volume×ventilation×Oasis® Horticubes® foam type. Data presented are as significant when prob. >F≦0.05.

### Results (Laboratory)

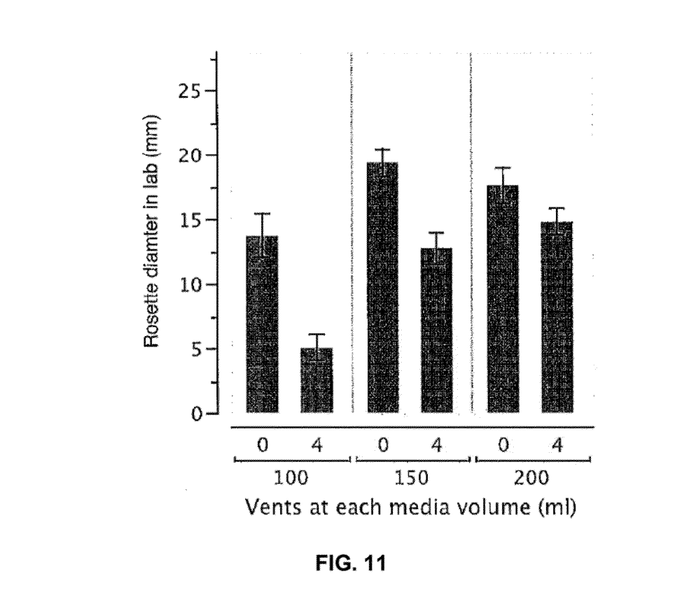

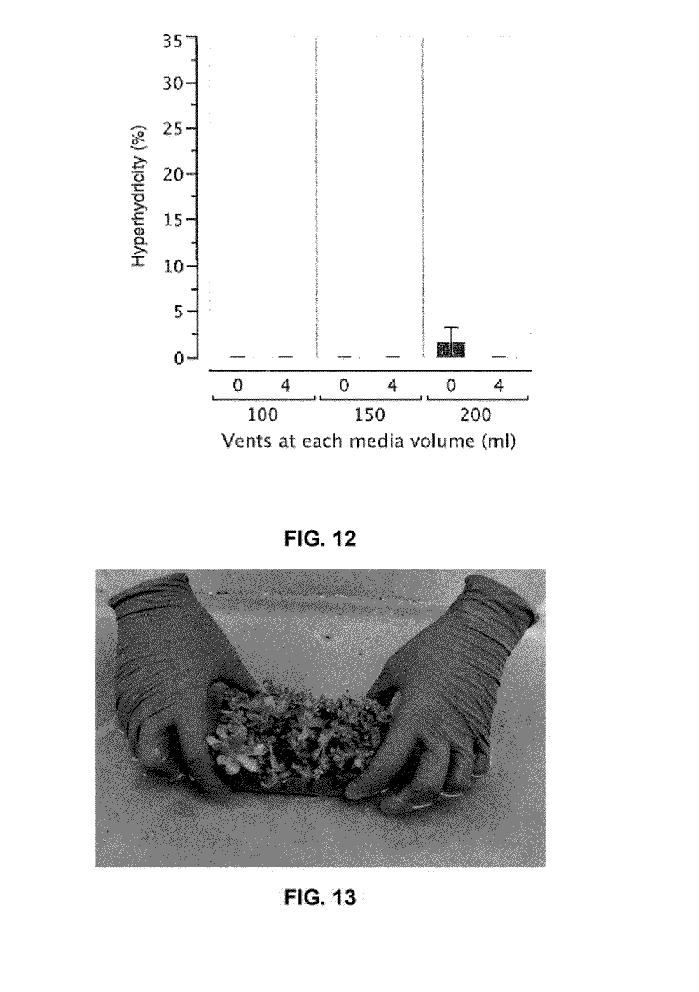

FIG. 10 shows Echeveria ‘Black Prince’ grown in a system including 150 mL of the liquid medium infused into Oasis® Horticubes® XL foam type over the 6-week micropropagation period. As can be seen, the plants grew well. Lower volumes of medium (100 ml) and the use of ventilated vessels produced smaller plants as measured by the diameter of the rosette (FIG. 11). Ventilation helped reduce hyperhydricity. In Oasis® Horticubes® XL foam, plant size increased with media volume in the 100-150 ml range, but did not increase significantly at greater volumes. Hyperhydricity was virtually nonexistent, and could be eliminated by either correct media volume, or ventilation (FIG. 12). Ventilation produced smaller plants than the non-vented vessels in all treatments. All plants were moved to greenhouse following the six week laboratory growth period.

### Results (Greenhouse)

Laboratory nutrient medium containing sucrose and other organic compounds was rapidly removed by rinsing the Oasis® Horticubes® XL growing medium, as shown in FIG. 13. Advantageously, all thirty-five plants of a single system could be handled at one time without damaging the roots. This is a major advantage over agar-solidified medium. The cells of the block were divided so single rooted plants (FIG. 14) could be planted in soilless medium. Having the root systems inserted into the growing medium without damage was another advantage of this system, and allowed the common procedure of rooting plants in greenhouse under mist to be skipped.

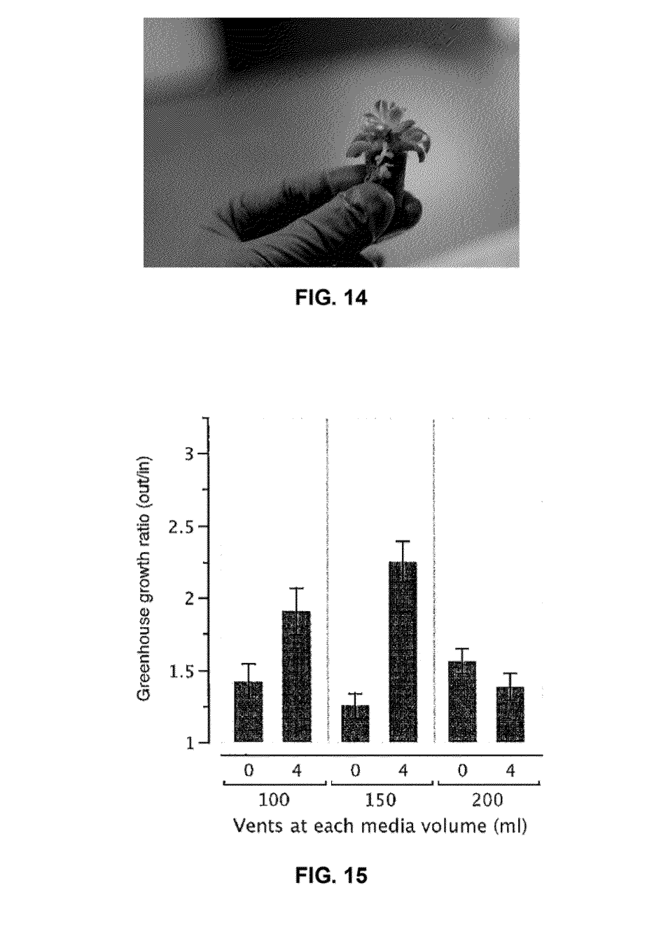

Following 5 weeks of greenhouse growth in full sun, non-hyperhydric plants survived at a rate of 95%, where the hyperhydric plants survived at a rate of 40%-70% (95% confidence interval). The greenhouse growth ratios are shown in FIG. 15. Plants from all laboratory treatments increased in size at rates that favorably compares to agar. The plants from the ventilated vessels increased in size more quickly than the sealed vessels. The large plants from 200 ml were not in as good condition to grow in the greenhouse and increase by 25% or 40%, for non- and ventilated treatments, respectively. With less medium volume (100-150 ml) plants could double in size, and since plants from 150 ml were large when they entered the greenhouse, this was an optimal treatment. In Oasis® Horticubes® XL foam treatment, plants from the non-ventilated vessels increased in size by about 45%, regardless of media volume. Plants from ventilated vessels with 150 ml treatment more than doubled in size (another optimal treatment), with less growth from 100 and 200 ml treatments, (1.8 and 1.4×, respectively).

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

1. A system for plant development comprising:
a container, the container including a base having a flat outer surface such that the container is self-standing, the base having dimensions that define the size of the base, the container having a flexible wall that is semi-permeable, the container wall and base being impermeable to biological microorganisms; and
a support within the interior of the container, the support being formed of a rigid or semi-rigid material of fixed dimension for supporting developing plant tissue;
the system further optionally comprising a growth medium.

2. The system of claim 1, wherein the container base includes a gusset.

3. The system of claim 1, wherein the container wall has a carbon dioxide permeability that is greater than or equal to about 100 cc/100 in²/24 hr at 1 atmosphere and has an oxygen permeability that is greater than or equal to about 100 cc/100 in²/24 hr at 1 atmosphere.

4. The system of claim 1, wherein the container defines a pore size that is about 0.2 micrometers or less.

5. The system of claim 1, wherein the container wall comprises polypropylene or high density polyethylene.

6. The system of claim 1, the container comprising one or both of a gas diffusion vent and a port in the container wall.

7. The system of claim 1, wherein the support comprises a polymeric foam.

8. The system of claim 1, wherein the support is segmented to form a plurality of individual cells, each of the cells optionally including one or more openings to receive plant tissue.

9. The system of claim 1, wherein the support has dimensions defining a surface of the support, the dimensions being from about 95% to about 99.9% of the dimensions of the base that define the size of the base.

10. The system of claim 1, wherein the interior of the container and the support are sterile.

11. A method of plant development comprising:
sterilizing the system of claim 1;
placing plant material in or on the support, the plant material including totipotent plant cells;
sealing the container with the plant material held within the container and in or on the support;
maintaining the system under conditions conducive to the growth and development of the plant material held within the system.

12. The method of claim 11, wherein the system is maintained in a laboratory, the method further comprising moving the system to a greenhouse or other cultivation environment from the laboratory.

13. The method of claim 11, further comprising transplanting the plant material following the growth and development of the plant material held within the system, the plant material being transplanted into soil in conjunction with the support.

14. The method of claim 11, further comprising intermittently misting the exterior of the container during the growth and development of the plant material.

15. The method of claim 11, wherein the plant material comprises a vegetative bud, bulbet, microtuber, callus, or somatic embryo or is derived from a vegetative bud, bulbet, microtuber, callus, genetic transformation, plant re-genesis, micropropagation, or somatic embryogenesis.

16. The system of claim 1, wherein the entire container is flexible.

17. The system of claim 1, wherein the container is translucent.

18. The system of claim 1, wherein the container wall has a moisture vapor transmission rate less than or equal to about 1 g/100 in²/24 hr at 1 atmosphere.

19. The system of claim 1, wherein the container wall comprises biaxially oriented polypropylene.

20. The system of claim 1, wherein the support comprises, a phenolic-based foam having a water holding capacity.

21. The method of claim 11, the method further comprising shipping the system following sealing of the container with the plant material held within the container.
